(19) United States
(12) Patent Application Publication
(10) Pub. No.: US 2026/0345933 A1
(43) Pub. Date: Nov. 5, 2026

(54) CONTROL UNIT FOR A MEDICAL VENTILATOR

(71) Applicant: Loewenstein Medical Technology S.A., Luxembourg (LU)

(72) Inventor: Joerg MARSCHINKE, Ehlscheid (DE)

(21) Appl. No.: 19/665,051

(22) Filed: May 1, 2026

(30) Foreign Application Priority Data
May 5, 2025 (DE) 10 2025 001 510.0

(51) Int. Cl. A61M 16/00

(52) U.S. Cl. CPC A61M 16/024; A61M 16/0003; A61M 2016/0027; A61M 2202/0007; A61M 2202/02; A61M 2205/3331; A61M 2205/50; A61M 2230/005; A61M 2230/432

(57) ABSTRACT

A ventilator comprises an actuation system for generating a respiratory air flow, a respiratory air connection for providing the respiratory air flow and a sensor system for recording at least one ventilation parameter with respect to a ventilation of a patient with the respiratory air flow provided by the respiratory air connection, the at least one ventilation parameter comprising a respiratory air pressure applied to the respiratory air connection and/or a respiratory air volume inhaled and/or exhaled by the patient. A control unit is configured to carry out a control method as set forth in the claims.

### CROSS-REFERENCE TO RELATED APPLICATIONS

The present application claims priority under 35 U.S.C. § 119 of German Patent Application No. 10 2025 001 510.0, filed May 5, 2025, the entire disclosure of which is expressly incorporated by reference herein.

### BACKGROUND OF THE INVENTION

### 1. Field of the Invention

The invention relates to a control unit for a ventilator. The invention also relates to a computer program that is executable by such a control unit, a corresponding computer- readable medium and a ventilator having such a control unit.

### 2. Discussion of Background Information

For example, in the case of invasive ventilation of a patient, the ventilation pressure can be controlled in such a way that the patient's lungs are stabilized in several stabilization phases while at the same time allowing spontaneous respiration by the patient. The stabilization phases can be interrupted by shorter relief phases, in which the ventilation pressure is significantly lower than in the previous stabilization phase, in order to effect (or enable) a correspondingly increased exhalation and thus temporarily relieve the pressure on the patient's lungs. Such a ventilation mode can also be called Airway Pressure Release Ventilation, APRV for short. APRV may cause the patient to be more or less sedated in the stabilization phases due to reduced exhalation of carbon dioxide, without the need for medication to be administered to the patient. Such carbon dioxide-related sedation or somnolence may be desirable under certain circumstances, but usually requires close monitoring of the patient by nursing staff.

In view of the foregoing, it would be advantageous to have available a control unit that allows improved control of carbon dioxide exhalation when ventilating a patient, for example in an invasive APRV, and thus improved control of carbon dioxide-related sedation of the patient. It would also be advantageous to have available a corresponding computer program, a corresponding computer-readable medium and a corresponding medical ventilator.

### SUMMARY OF THE INVENTION

In a first aspect, the invention provides a a control unit for a ventilator.

The ventilator comprises an actuation system for generating a respiratory air flow, a respiratory air connection for providing the respiratory air flow and a sensor system for recording at least one ventilation parameter with respect to a ventilation of a patient with the respiratory air flow provided by the respiratory air connection. The one or more ventilation parameter comprises a respiratory air pressure applied to the respiratory air connection (during patient ventilation) and/or a respiratory air volume inhaled and/or exhaled by the patient (during patient ventilation).

The control unit is configured to carry out the following control method: receiving sensor data generated by means of the sensor system, which indicate a (for example time-dependent) actual course of the at least one ventilation parameter; generating a control signal for controlling the actuation system using the sensor data to adjust the respiratory air flow so that the actual course approaches a target course. The target course comprises a plurality of stabilization phases, in which the actual course is intended to approach an upper limit (for the respective ventilation parameter) and a plurality of relief phases, in which the actual course is intended to approach a lower limit (located below the upper limit) (for the respective ventilation parameter). The stabilization phases and the relief phases alternate with each other. The upper limit is defined by a reference value for stabilizing the patient's lungs and to allow the patient to breathe spontaneously. The upper limit is further varied by repeatedly and alternately increasing and decreasing its value in relation to the reference value in each stabilization phase in order to effect additional carbon dioxide exhalation in the respective stabilization phase. The additional carbon dioxide exhalation can be significantly greater than a normal carbon dioxide exhalation for a constant upper limit in the respective stabilization phase. The lower limit is defined in its value relative to the reference value in each relief phase such that in the respective relief phase, an increased exhalation in comparison to a stabilization phase (immediately) preceding the respective relief phase is effected (or at least enabled) to relieve the patient's lungs.

Such a control unit allows the carbon dioxide exhalation – and thus a carbon dioxide-related sedation or somnolence of the patient that may be desired in some circumstances – to be controlled in a targeted manner, for example automatically, during each stabilization phase. In other words, such a control unit enables a targeted control of carbon dioxide exhalation – with an appropriately adjusted APRV – throughout the entire ventilation period, not only during the relief phases. In this way, excessively high carbon dioxide concentrations in the patient's respiratory air and/or blood can be effectively reduced for a short period by a suitably adjusted exhalation, without the frequency of the relief phases or a temporal ratio between the inhalation and exhalation phases needing to be changed. In some cases, extracorporeal membrane oxygenation, or ECMO for short, can even be dispensed with as a possible last treatment measure. Another advantage is that, due to the targeted control of the intensity of carbon dioxide-related sedation, no or significantly smaller amounts of sedative drugs can be administered. This can prevent or at least alleviate undesirable side effects.

The control unit may comprise means for data processing. The means for data processing may be implemented as hardware and/or software and/or may comprise a processor. The processor may be configured to carry out the (computer-implemented) control method. In addition to the processor, the control unit may comprise at least one of the following data-processing means: a memory, a bus system for communicating data between the memory and the processor, and a data communications interface for communicating data with peripherals wirelessly and/or in wired fashion. Alternatively, the control unit can be implemented exclusively as hardware, for example in the form of an ASIC component or FPGA component.

"Sensor data" can preferably be understood as digital data that encodes the actual course in a suitable manner. The sensor data can be, for example, output data of the sensor system and/or processed data generated by processing a (raw, for example analog) output signal of the sensor system. It is possible that the sensor data is stored (or cached) in the memory of the control unit and can be received from there.

“Actual course” can be understood as a measured and/or estimated (for example, time-dependent) course of the respective ventilation parameter. In other words, the sensor data may comprise a time series of (measured and/or estimated) actual values of the respective ventilation parameter as the actual course.

In a corresponding manner, the target course can be considered as a time series of (specified) setpoints for the respective ventilation parameter. The target course may be stored in the memory of the control unit, for example in the form of a mathematical function and/or a lookup table.

It is possible that each stabilization phase will immediately transition to one of the relief phases (or vice versa). Expediently, each relief phase can be significantly shorter than the preceding stabilization phase. For example, each relief phase can last for only a single exhalation or single breath and/or no longer than 10s, 5s, 2s, or 1s. On the other hand, each stabilization phase may comprise multiple breaths and/or last for at least 2s or at least 4s. In some cases, for example, if the patient is a newborn or preterm baby, an infant or a baby, each relief phase can even last significantly less than 1s, preferably between 0.3s and 0.6s. In these cases, for example, each stabilization phase can last significantly less than 2s.

The upper limit may correspond, for example, to a target pressure, preferably a specified positive end-expiratory pressure, and/or to a target volume, preferably a specified tidal volume, for stabilizing the lungs of the patient and for enabling the patient to breathe spontaneously. In other words, a suitable upper limit allows the patient to spontaneously inhale and/or exhale during each stabilization phase if he/she is able to do so. The lower limit can correspond to a significantly lower target pressure or target volume compared to the upper limit for relieving the patient's lungs. For example, the lower limit can be between 10% and 30% of the upper limit (or the reference value).

The upper limit may vary in a regular manner (for example, according to a specified frequency) and/or irregularly (for example, depending on a more or less uniform spontaneous respiration of the patient).

It is possible that during its variation the upper limit may be increased and/or decreased relative to its current value either in one stage or in multiple stages, for example, according to a suitable linear or non-linear mathematical function and/or depending on a respective inhalation duration. Such a mathematical function can also be referred to as a ramp function or ramp for short, and/or be stored as a lookup table in the memory of the control unit.

The upper limit can be expediently set to the reference value at the beginning and at the end of each stabilization phase (for example, in the first time step and in the last time step of each stabilization phase).

The upper limit can also be referred to as a respiratory plateau. The lower limit can also be referred to as the relief pressure or relief volume, depending on the ventilation parameter.

The value of the lower limit relative to the reference value may also be defined in such a way that the patient is also able to breathe spontaneously in each relief phase. In either case, the lower limit should be defined in such a way as to avoid partial or complete collapse of the patient's lungs in each relief phase.

For example, the reference value may correspond to a pressure or volume at which the patient's lungs are as fully deployed as possible. The reference value may have been determined empirically, for example, by adjusting the reference value taking into account the actual deployment of the patient's lungs (as a function of the reference value) during their ventilation, by means of an appropriate imaging method (e.g. by means of an X-ray).

Depending on the patient's condition or the ventilation situation, the reference value defining the upper limit in a current stabilization phase may differ (significantly) from the reference value defining the upper limit in at least one previous stabilization phase (i.e. preceding the current stabilization phase). The same may apply analogously to the value of the lower limit in a current relief phase.

In a second aspect, the invention provides a ventilator for invasive and/or non-invasive ventilation of a patient. The ventilator comprises an actuation system for generating a respiratory air flow, a respiratory air connection for providing the respiratory air flow and a sensor system for recording at least one ventilation parameter with respect to the ventilation of the patient with the respiratory air flow provided by the respiratory air connection, and a control unit as described above and below. The one or more ventilation parameter comprises a respiratory air pressure applied to the respiratory air connection (during patient ventilation) and/or a respiratory air volume inhaled and/or exhaled by the patient (during patient ventilation).

The term "ventilator" can also mean an anesthesia machine.

The respiratory air connection can be connected to the patient via at least one respiratory air line, so that the patient can be ventilated with the respiratory air flow. For example, "respiratory air line" can mean a breathing tube and/or a suitable patient interface such as a mask, nasal cannula, or tube.

The actuation system may comprise one or more electropneumatic actuators for generating the respiratory air flow. Such an actuator may be, for example, a blower or an electrically controllable valve and/or be controllable by the control unit.

The sensor system may comprise one or more sensors for acquiring the respective ventilation parameter. Such a sensor may be, for example, a pressure sensor for recording the respiratory air pressure, a flow sensor for recording a volume flow of the respiratory air inhaled and/or exhaled by the patient, or a gas sensor for recording a partial pressure of a respiratory gas (for example, carbon dioxide or oxygen) in air inhaled and/or exhaled by the patient and/or in the blood of the patient. Depending on the embodiment, such a sensor may be arranged in a primary or secondary stream of the respiratory air inhaled and/or exhaled by the patient.

In a third aspect, the invention povides a computer program for operating the ventilator described above and below. The computer program comprises commands that cause the ventilator control unit, such as a processor of the control unit, to carry out the following (computer-implemented) control method upon execution of the computer program by the control unit (or processor).

In a fourth aspect, the invention povides a computer-readable medium on which the computer program described above and below is stored.

The computer-readable medium may be a transitory or non-transitory data medium. For example, the computer-readable medium may be a hard disk, a USB (universal serial bus) storage device, a RAM (random-access memory), a ROM (read-only memory), an EPROM (erasable programmable read-only memory), an EEPROM (electrically erasable programmable read-only memory), a flash memory or a combination of at least two of these examples. The computer-readable medium may also be a data communications network that allows program code to be downloaded (e.g. via the Internet), or a cloud.

It should be noted that features of the control unit described above and below can also be features of the computer program and/or the computer-readable medium (and vice versa).

Various embodiments are described below. These embodiments should not be understood as restricting the scope of the disclosure.

According to one embodiment, the control method may further comprise evaluating the sensor data to detect an actual spontaneous respiration of the patient in one of the stabilization phases. Alternatively, the control method may also comprise: receiving volume flow data generated by means of the sensor system, which indicate a (for example, time-dependent) course of a volume flow of respiratory air inhaled and/or exhaled by the patient (during their ventilation), and evaluating the volume flow data alone or in combination with the sensor data in order to detect actual spontaneous respiration of the patient in one of the stabilization phases. In both cases, if the actual spontaneous respiration is detected, the upper limit in the respective stabilization phase can be varied depending on the actual spontaneous respiration detected, for example, depending on a spontaneous respiration frequency at which the patient is breathing spontaneously. The actual spontaneous respiration can be detected, for example, by comparing the actual course of the at least one ventilation parameter and/or the course of the volume flow with a corresponding (for example empirically determined) reference course, which is characteristic of spontaneous inhalation or exhalation.

According to one embodiment, if spontaneous inhalation is recognized as the actual spontaneous respiration, the upper limit can be set to a peak value. The peak value can be greater than the reference value by a specified amount. For example, the peak value may be equal to a sum of the reference value and a value of a specified amplitude as the specified amount.

In addition or alternatively, if spontaneous exhalation is recognized as the actual spontaneous respiration, the upper limit can be set to a valley value. The valley value can be equal to the reference value, or be less than the reference value by a specified amount. For example, the valley value may be equal to a difference between the peak value and a value of a specified amplitude as the specified amount.

According to one embodiment, the control unit can be switched between a first variation mode and a second variation mode. In this case, it is possible that, when the first variation mode is activated, the upper limit is varied depending on the patient's spontaneous respiration and/or, when the second variation mode is activated, the upper limit is varied independently of the spontaneous respiration of the patient.

For example, when the first variation mode is activated, the upper limit can be varied taking into account the detected actual spontaneous respiration (for example, according to a detected spontaneous respiration frequency) and according to a specified amplitude. In addition or alternatively, if the second variation mode is activated, the upper limit can be varied according to a specified frequency or according to a specified frequency and a specified amplitude. Any detected actual spontaneous respiration can be ignored.

Expediently, the control unit may be configured to prevent operation in the second variation mode when the first variation mode is activated, and vice versa.

For example, in each stabilization phase the first variation mode can be a combination of an APRV mode and a PSV (Pressure Support Ventilation) mode.

For example, in each stabilization phase the second variation mode can be a combination of an APRV mode with an HFO (High Frequency Oscillation) mode.

According to one embodiment, the upper limit can be varied according to a specified frequency and/or a specified amplitude.

Varying the upper limit according to the specified amplitude can comprise, for example, increasing the upper limit by adding a value of the specified amplitude to a current value of the upper limit (for example, to the reference or valley value) and/or decreasing in the upper limit by subtracting a value of the specified amplitude from a current value of the upper limit (for example, from the reference or peak value).

For example, the upper limit can be varied either according to the specified frequency or according to the specified frequency and the specified amplitude as long as (for example, in the first variation mode) no actual spontaneous respiration of the patient is detected in the respective stabilization phase and/or when the second variation mode is activated.

According to one embodiment, the method may further comprise: receiving partial pressure data generated by means of the sensor system, comprising one or more partial pressure values and/or a time-dependent course of a carbon dioxide partial pressure (and/or a partial pressure of at least one other respiratory gas such as oxygen, nitrogen or anesthetic gas) in a respiratory air exhaled by the patient and/or in the blood of the patient; determining the specified frequency using the partial pressure data and a frequency assignment rule that assigns a specific frequency to possible partial pressure values. In addition or alternatively, the control method may comprise: determining the specified amplitude using the partial pressure data and an amplitude assignment rule that assigns a specific amplitude to possible partial pressure values.

This makes it possible to automatically control the carbon dioxide respiration in each stabilization phase depending on an actual carbon dioxide partial pressure in the respiratory air exhaled by the patient or in the blood of the patient, for example, by accordingly increasing the specified frequency or amplitude in its value at an excessively high actual carbon dioxide partial pressure, in order to effect a correspondingly increased carbon dioxide exhalation (and thus a corresponding reduction in the actual carbon dioxide partial pressure) and/or at an excessively low actual carbon dioxide partial pressure, in order to effect a correspondingly reduced carbon dioxide exhalation (and thus a corresponding increase in the actual carbon dioxide partial pressure). With the aid of such control, the actual carbon dioxide partial pressure can quickly and accurately approach a desired carbon dioxide partial pressure. This has the advantage that the effort involved in monitoring the patient by nursing staff can be significantly reduced in comparison to an embodiment without such control.

For example, an "assignment rule" such as a "frequency assignment rule" or "amplitude assignment rule" can be understood as a mathematical function or lookup table. Such an assignment rule can be stored, for example, in the memory of the control unit. Depending on the partial pressure value, the specified frequency and/or the specified amplitude can also be zero.

According to one embodiment, the control method can further comprise determining the specified frequency and/or the specified amplitude taking into account the spontaneous respiration of the patient, for example according to a spontaneous respiration frequency at which the patient is breathing spontaneously.

According to one embodiment, the specified frequency can be from about 5 Hz to about 25 Hz, preferably from about 10 Hz to about 20 Hz. Such a frequency range has proved to be particularly effective in practice.

According to one embodiment, the specified amplitude can correspond to a pressure difference of from about 2 mbar to about 15 mbar, preferably of at least 3 mbar and/or at most 10 mbar. Such a pressure difference range has proved to be particularly effective in practice.

According to one embodiment, the method may further comprise: receiving volume flow data generated by the sensor system, which indicate a (for example time-dependent) course of a volume flow of respiratory air exhaled by the patient in a current relief phase; determining a transition time at which the current relief phase should transition to a new stabilization phase, using the volume flow data; switching the target course from the lower limit to the upper limit at the transition time, so that the current relief phase transitions to the new stabilization phase. This can effectively prevent a complete emptying and thus a partial or complete collapse of the patient's lungs, in particular the alveoli, when exhaling in the current relief phase.

In other words, in order to further safeguard the value set for the lower limit, the control unit or the control method may comprise a special flow control function, which is oriented toward an expiratory flow profile and, for example, controls the volume flow or flow using a suitable percentage. This makes it possible to flexibly adapt the relief phases to sudden changes in compliance and resistance during ventilation, for example as a result of a patient relocation.

According to one embodiment, the course of the volume flow can be compared with a threshold value. A time at which the course of the volume flow reaches the threshold value (for the first time) can be determined as the transition time. For example, the threshold may be from about 10% to about 70% or from about 20% to about 50%, preferably about 25%, of a maximum of the volume flow of the respiratory air exhaled by the patient in a single breath.

According to one embodiment, the upper limit can correspond to a positive end-expiratory pressure from about 5 mbar to about 60 mbar, from about 5 mbar to about 40 mbar, from about 5 mbar to about 30 mbar, from about 5 mbar to about 25 mbar, or from about 5 mbar to about 20 mbar. In this case, the reference value may be smaller than the highest value of the above-mentioned value ranges by a specified amount, which may, for example, be from about 2 mbar to about 15 mbar, preferably from about 3 mbar to about 10 mbar. For example, the maximum value of these ranges can be the highest permissible value and/or the peak value.

According to one embodiment, the lower limit can correspond to a positive end-expiratory pressure of from 0 mbar to about 30 mbar or from 0 mbar to about 5 mbar. In addition or alternatively, the lower limit can be constant in each relief phase.

According to one embodiment, a temporal ratio of the stabilization phases to the relief phases relative to a suitable reference duration can be at least two to one or at least four to one. In other words, each stabilization phase can be significantly longer than the (immediately) subsequent relief phase, for example at least twice or at least four times as long as the (immediately) subsequent relief phase.

According to one embodiment, each stabilization phase can comprise at least two or at least four breaths. In other words, each stabilization phase can be at least long enough to allow at least two or at least four immediately consecutive (spontaneous) breaths during the stabilization phase.

### BRIEF DESCRIPTION OF THE DRAWINGS

In the following, exemplary embodiments are described with reference to the accompanying drawings. Neither the description nor the drawings should be understood as restricting the scope of the invention. In the drawings:

FIG. 1 shows an exemplary embodiment of a ventilator.

FIG. 2 shows a time-dependent actual course of ventilation parameters, which can be controlled by a control unit from FIG. 1 according to a target course with alternating stabilization and relief phases, wherein the target course in each stabilization phase is varied depending on a spontaneous respiration.

FIG. 3 shows a time-dependent actual course of ventilation parameters, which can be controlled by a control unit from FIG. 1 according to a target course with alternating stabilization and relief phases, wherein a high-frequency oscillation independent of spontaneous respiration is used as the target course in each stabilization phase.

The drawings are purely schematic and not true to scale. Where identical reference signs are used in different drawings, these reference signs denote identical or identically acting features.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

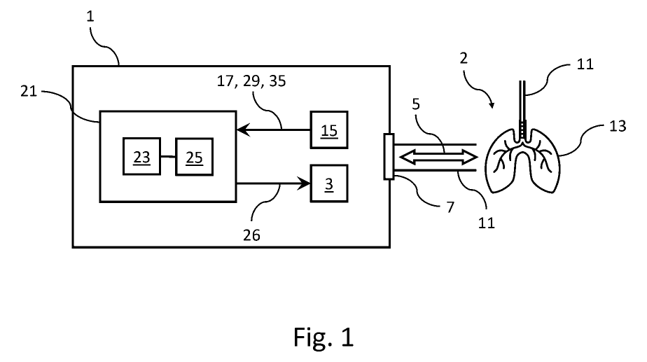

FIG. 1 shows a ventilator 1 for invasive ventilation of a patient 2. The ventilator 1 comprises an actuation system 3 for generating a respiratory air flow 5 and a respiratory air connection 7 for providing the respiratory air flow 5. The respiratory air connection 7 is connected via one or more suitable respiratory air lines 11, for example, one or more breathing tubes and a tube, to the respiratory apparatus of the patient 2, more precisely to the lung 13 of the patient 2, so that the patient 2 can be ventilated with the respiratory air flow 5.

In addition or alternatively, the ventilator 1 may be suitable for non-invasive ventilation of the patient 2, for example via an oral, nasal or face mask or a nasal cannula.

The actuation system 3 can comprise one or more electropneumatic actuators for generating the respiratory air flow 5, for example, one or more blowers and/or one or more electrically controllable valves.

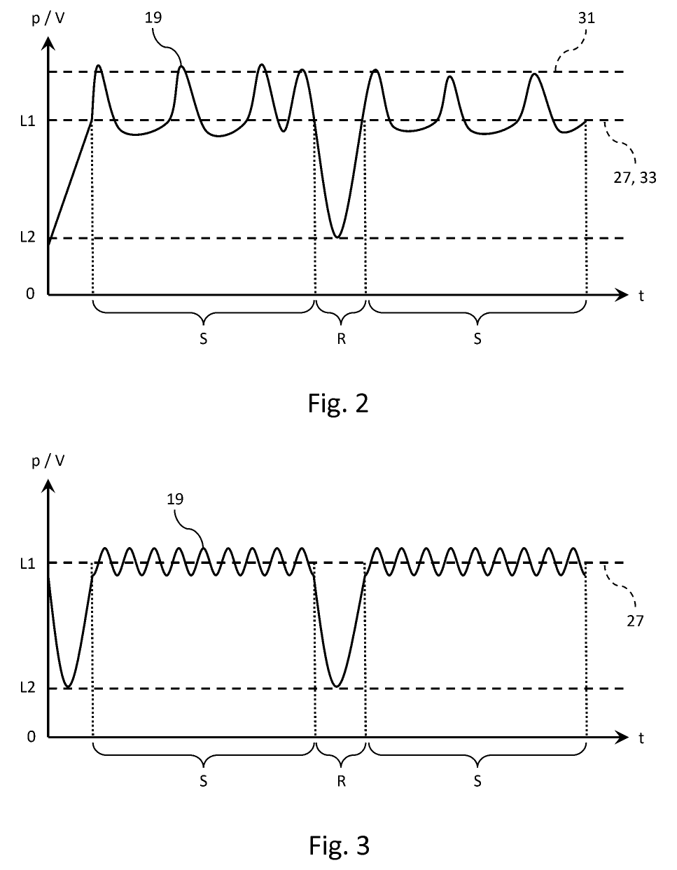

In addition, the ventilator 1 comprises a sensor system 15 for recording at least one ventilation parameter with respect to the ventilation of the patient 2, in this case a respiratory air pressure p applied to the respiratory air connection 7 (see FIGS. 2 and 3). Accordingly, the sensor system 15 may comprise one or more pressure sensors for recording the respiratory air pressure p. The sensor system 15 is designed to generate and output corresponding sensor data 17. In this example, the sensor data 17 encodes a time-dependent actual course 19 of the respiratory air pressure p during ventilation of the patient 2.

In addition or alternatively, the sensor system 15 may be designed to record a respiratory air volume V (see FIGS. 2 and 3) inhaled and/or exhaled by the patient 2 as the ventilation parameter. For this purpose, the sensor system 15 may comprise one or more flow sensors for recording a volume flow of a respiratory air inhaled and/or exhaled by the patient 2. For example, the respiratory air volume V can be determined by integrating the measured volume flow. Such a flow sensor may be arranged in a primary or secondary stream of the respiratory air inhaled and/or exhaled by the patient 2.

In addition, the ventilator 1 comprises a control unit 21 for controlling the actuation system 3. The control unit 21 can comprise a processor 23 and a memory 25. The memory 25 can contain a computer program for operating the ventilator 1. By executing the computer program, the processor 23 can be configured to carry out a control method for controlling the respiratory air flow 5. For example, the control method may be performed continuously (and/or in multiple successive time steps) during ventilation of the patient 2 or during operation of the ventilator 1.

In a first step of the control method, the sensor data 17 is received.

In a second step of the control method, a control signal 26 for controlling the actuation system 3 is generated using the sensor data 17 to adjust the respiratory air flow 5 such that the actual course 19 approaches a target course between an upper limit L1 and a lower limit L2 for the respective ventilation parameter, here the respiratory air pressure p. Depending on the embodiment, the target course may also refer to the respiratory air volume V or another suitable ventilation parameter.

The target course comprises several stabilization phases S, in which the actual course 19 is intended to approach the upper limit L1 in each case, and a plurality of relief phases R, in which the actual course 19 is intended to approach the lower limit L2 in each case (in FIGS. 2 and 3 only one of the relief phases R is shown by way of example). The stabilization phases S can alternate with the relief phases R. Each stabilization phase S can immediately transition to one of the relief phases R and/or each relief phase R can immediately transition to one of the stabilization phases S.

The upper limit L1 is defined by a reference value 27 for stabilizing the lungs 13 of the patient 2 and to allow the patient 2 to breathe spontaneously. In this example, the reference value 27 is a suitable pressure value, for example a suitable value of a positive end-expiratory pressure (PEEP for short).

The lower limit L2 is defined in its value relative to the reference value 27 in such a way that in each relief phase R an increased exhalation in comparison to the respective immediately preceding stabilization phase S is effected (or at least made possible) in order to relieve the lungs 13 of the patient 2. A corresponding amount of carbon dioxide is exhaled. The lower limit L2 can be selected in such a way that in such a way that the patient 2 is also able to breathe spontaneously in each relief phase R. Generally speaking, the lower limit L2 can be smaller in magnitude than the upper limit L1. In this example, the upper limit L1 corresponds to a higher positive end-expiratory pressure and the lower limit L2 to a significantly lower positive end-expiratory pressure compared to the upper limit L1.

In order to improve carbon dioxide exhalation in the stabilization phases S, the upper limit L1 is varied in each stabilization phase S by repeatedly increasing and decreasing its value alternately in relation to the reference value 27. This may be dependent on, or independent of, any spontaneous respiration of the patient 2.

In this case, a temporal ratio of the stabilization phases S to the relief phases R relative to a reference duration may be at least two to one, for example three to one, four to one, five to one, six to one or more, in some cases even ten to one (or more). For example, the reference duration may be 5s, 10s, 1 min, or between 1 min and 10 min, or equal to a (average) duration of a single breath or multiple (immediate) consecutive breaths. Such a temporal ratio may, for example, be specified or result from a (for example specified) frequency with which the stabilization phases S are to be interrupted by a relief phase R.

Expediently, the stabilization phases S may each comprise at least two or at least four (immediately consecutive) breaths. However, stabilization phases S are also possible which in each case comprise only a single breath or only a single inhalation or exhalation phase of a single breath.

In addition, the sensor data 17 can be evaluated to detect actual spontaneous respiration of the patient 2 in one of the stabilization phases S.

Alternatively, volume flow data 29 generated by means of the sensor system 15 (for example, by means of the flow sensor or flow sensors) can be received, which indicate a (for example time-dependent) course of a volume flow of respiratory air inhaled and/or exhaled by the patient 2. In this case, it is possible that the volume flow data 29 are evaluated alone or in combination with the sensor data 17 in order to detect actual spontaneous respiration of the patient 2 in one of the stabilization phases S.

In both cases, if the actual spontaneous respiration is detected, the upper limit L1 in the respective stabilization phase S can be varied depending on the actual spontaneous respiration (see FIG. 2) detected, for example depending on a spontaneous respiration frequency at which the patient 2 is breathing spontaneously, and/or depending on a detected spontaneous inhalation and/or exhalation of the patient 2.

For example, whenever spontaneous inhalation is recognized as actual spontaneous respiration, the upper limit L1 can be set to a peak value of 31 that is greater than the reference value of 27 by a specified amount.

In addition or alternatively, when spontaneous exhalation is detected as the actual spontaneous respiration, the upper limit L1 can be set to a valley value of 33 which is equal to the reference value 27. In some cases, the valley value 33 may also be less than the reference value 27 by a specified amount. In other words, depending on the actual spontaneous respiration detected, the target course can be alternately switched between the peak value 31 and the valley value 33 (or between the peak value 31 and the reference value 27).

It is possible that the control unit 21 can be switched between a first variation mode and a second variation mode. In this case, when the first variation mode is activated, the upper limit L1 can be varied depending on a (detected) spontaneous respiration of the patient 2 and, when the second variation mode is activated, the upper limit L1 is varied independently of a (detected) spontaneous respiration of patient 2.

For example, if the first variation mode is activated, the upper limit L1 can also be varied according to a specified amplitude (here a specified pressure difference). In this case, a frequency at which the upper limit L1 is varied in its value relative to the reference value 27 can be specified as a function of a spontaneous respiration frequency at which the patient 2 breathes spontaneously.

In addition or alternatively, if the second variation mode is activated, the upper limit L1 can be varied according to a specified frequency and a specified amplitude (here a specified pressure difference). Any detected spontaneous respiration of the patient 2 can then be ignored.

The specified amplitude can correspond, for example, to a pressure difference between 2 mbar and 15 mbar, preferably of at least 3 mbar and/or at most 10 mbar, depending on the condition of the patient 2 or depending on the ventilation situation.

For example, the specified frequency can be from about 5 Hz to about 25 Hz, preferably from about 10 Hz to about 20 Hz. A periodic variation of the upper limit L1 at such a frequency can also be referred to as high-frequency oscillation, or HFO for short (see FIG. 3).

For example, provided (for example, in the first variation mode) no actual spontaneous respiration of the patient 2 is detected in the respective stabilization phase S and/or when the second variation mode is activated, the upper limit L1 can be varied either according to the specified frequency or according to the specified frequency and the specified amplitude.

For example, in each stabilization phase S the first variation mode can be a combination of an APRV mode and a PSV mode.

For example, in each stabilization phase S the second variation mode can be a combination of an APRV mode and an HFO mode.

Conveniently, the control unit 21 may be configured to prevent operation in the second variation mode when the first variation mode is activated and/or to prevent operation in the first variation mode when the second variation mode is activated.

For example, the upper limit L1 may correspond to a positive end-expiratory pressure of from about 5 mbar to about 60 mbar. In other words, the actual course 19 should not exceed a pressure value of 60 mbar when varying in each stabilization phase S. The reference value 27 should therefore be selected to be lower than this maximum permissible pressure value by an appropriate amount, for example by the amount of the specified amplitude.

Depending on the respective upper limit L1, the lower limit L2 in this case can correspond, for example, to a positive end-expiratory pressure of from 0 mbar to 30 mbar. The lower limit L2 can also be constant in each relief phase R, i.e. have the same value during the entire relief phase R.

The specified frequency and/or amplitude to vary the upper limit L1 can also be determined automatically.

For example, the sensor system 15 for this purpose can comprise one or more gas sensors for detecting a carbon dioxide partial pressure in respiratory air exhaled by a patient 2 and/or in the blood of the patient 2.

In this case, in addition, partial pressure data 35 generated by means of the sensor system 15 (more precisely by means of the gas sensor or the gas sensors), which comprises one or more (for example chronologically consecutive) partial pressure values of the carbon dioxide partial pressure, can be received in the control unit 21.

In a further optional step, the partial pressure data 35 can be used in combination with a corresponding frequency assignment rule (for example, stored in the memory 25 as a lookup table or mathematical function), which assigns a specific frequency to possible partial pressure values in each case, in order to select a suitable frequency for the variation of the upper limit L1. The frequency selected in this way can then be used as the specified frequency.

In addition or alternatively, the partial pressure data 35 can be used in combination with a corresponding amplitude assignment rule (for example, stored in the memory 25 as a lookup table or mathematical function), which assigns a specific amplitude to possible partial pressure values in each case, in order to select a suitable amplitude for the variation of the upper limit L1. The amplitude selected in this way can then be used as the specified amplitude.

Depending on the partial pressure value, the specified frequency and/or the specified amplitude can also be zero.

This allows the carbon dioxide exhalation to be controlled automatically in each stabilization phase S as a function of an actual carbon dioxide partial pressure in the respiratory air exhaled by the patient 2 or in the blood of the patient 2.

In addition, the control unit 21 may be configured to control the duration of each relief phase R depending on respiratory air exhaled by the patient 2 in the respective relief phase R.

For example, the volume flow data 29, which indicate a course of a volume flow of a respiratory air exhaled by the patient 2 in a current relief phase R, can be used to determine a transition time at which the current relief phase R should transition to a new stabilization phase S. The course of the volume flow can then be compared with a suitable threshold value and a time when the course of the volume flow reaches the threshold value (for the first time) can be determined as the transition time. A suitable threshold may be, for example, correspond to a proportion of from about 10% to about 70% or from about 20% to about 40%, preferably about 25%, of a possible total volume flow of the respiratory air exhaled by the patient 2 during a single breath.

The target course can then be switched from the lower limit L2 to the upper limit L1 at the transition time, so that the current relief phase R transitions to the new stabilization phase S.

In this way, a partial or complete collapse of the lungs 13 of the patient 2, in particular of the alveoli, can be effectively prevented during exhalation in the respective relief phase R.

Finally, it should be noted that terms such as “have”, “comprise”, “include”, “having” etc. do not exclude other elements or steps, and indefinite articles such as “a” or “an” do not exclude a plurality.

Furthermore, it should be noted that features or steps that are described with reference to one of the embodiments above can also be used in combination with features or steps that are described with reference to other embodiments from among the embodiments above.

Reference signs in the claims should not be understood as restricting the scope of the subject matter defined by the claims.

### LIST OF REFERENCE SIGNS

1 Ventilator
2 Patient
3 Actuation system
5 Respiratory air flow
7 Respiratory air connection
11 Respiratory air line
13 Lungs
15 Sensor system
17 Sensor data
19 Actual course
21 Control unit
23 Processor
25 Memory
26 Control signal
27 Reference value
29 Volume flow data
31 Peak value
33 Valley value
35 Partial pressure data
P Respiratory air pressure
t Time
L1 Upper limit
L2 Lower limit
R Relief phase
S Stabilization phase
V Respiratory air volume

What is claimed is:

1. A control unit for a ventilator, the ventilator comprising:
an actuation system for generating a respiratory air flow;
a respiratory air connection for providing the respiratory air flow;
a sensor system for recording at least one ventilation parameter with respect to a ventilation of a patient with the respiratory air flow provided by the respiratory air connection, the at least one ventilation parameter comprising a respiratory air pressure (p) applied to the respiratory air connection and/or a respiratory air volume (V) inhaled and/or exhaled by the patient;
wherein the control unit is configured to carry out the following control method:
receiving sensor data generated by the sensor system, which indicate an actual course of the at least one ventilation parameter;
generating a control signal for controlling the actuation system using the sensor data in order to adjust the respiratory air flow such that the actual course approaches a target course, the target course comprising a plurality of stabilization phases (S), in which the actual course is intended to approach an upper limit (L1) in each case, and a plurality of relief phases (R), in which the actual course is intended to approach a lower limit (L2) in each case, wherein the stabilization phases (S) and the relief phases (R) alternate, the upper limit (L1) being defined by a reference value for stabilizing lungs of the patient and allowing the patient to breathe spontaneously and is varied by repeatedly and alternately increasing and decreasing its value in relation to the reference value, in order to effect an additional carbon dioxide exhalation in each stabilization phase, and the lower limit (L2) being defined in its value relative to the reference value in such a way that in each relief phase (R) an increased exhalation in comparison to a respective preceding stabilization phase (S) is effected to relieve the lungs of the patient.

2. The control unit of claim 1, wherein the control method further comprises:
evaluating the sensor data to detect actual spontaneous respiration of the patient in one of the stabilization phases (S), or
receiving volume flow data generated by the sensor system, which indicate a course of a volume flow of respiratory air inhaled and/or exhaled by the patient, and evaluating the volume flow data alone or in combination with the sensor data in order to detect actual spontaneous respiration of the patient in one of the stabilization phases (S);
wherein, when the actual spontaneous respiration is detected, the upper limit (L1) in the respective stabilization phase (S) is varied depending on the actual spontaneous respiration detected.

3. The control unit of claim 2,
wherein, when spontaneous inhalation is detected as the actual spontaneous respiration, the upper limit (L1) is set to a peak value that is greater than the reference value by a specified amount, and/or
wherein, when spontaneous exhalation is detected as the actual spontaneous respiration, the upper limit (L1) is set to a valley value which is equal to the reference value or less than the reference value by a specified amount.

4. The control unit of claim 1,
wherein the control unit can be switched between a first variation mode and a second variation mode,
wherein, when the first variation mode is activated, the upper limit (L1) is varied depending on the patient's spontaneous respiration,
wherein, when the second variation mode is activated, the upper limit (L1) is varied independently of the patient's spontaneous respiration.

5. The control unit of claim 4, wherein the upper limit (L1) is varied according to a specified frequency and/or a specified amplitude.

6. The control unit of claim 5, wherein, when the second variation mode is activated, the upper limit (L1) is varied at least according to the specified frequency.

7. The control unit of claim 5, wherein the method further comprises:
receiving partial pressure data generated by the sensor system, comprising one or more partial pressure values of a carbon dioxide partial pressure in a respiratory air exhaled by the patient and/or in blood of the patient;
determining a specified frequency using the partial pressure data and a frequency assignment rule that assigns a specific frequency to possible partial pressure values, and/or
determining a specified amplitude using the partial pressure data and an amplitude assignment rule that assigns a specific amplitude to possible partial pressure values.

8. The control unit of claim 7,
wherein the specified frequency is from 5 Hz to 25 Hz, and/or
wherein a specified amplitude corresponds to a pressure difference of from 2 mbar to 15 mbar.

9. The control unit of claim 1, wherein the control method further comprises:
receiving volume flow data generated by the sensor system, which indicate a course of a volume flow of respiratory air exhaled by the patient in a current relief phase (R);
determining a transition time at which the current relief phase should transition to a new stabilization phase (S), using the volume flow data;
switching a target course from the lower limit to the upper limit at the transition time, so that the current relief phase (R) transitions to a new stabilization phase (S).

10. The control unit of claim 9, wherein the course of the volume flow is compared with a threshold value and a time when the course of the volume flow reaches the threshold value is determined as the transition time.

11. The control unit of claim 1,
wherein the upper limit (L1) corresponds to a positive end-expiratory pressure of from 5 mbar to 60 mbar and/or
wherein the lower limit (L2) corresponds to a positive end-expiratory pressure of from 0 mbar to 30 mbar and/or is constant in each relief phase (R).

12. The control unit of claim 1,
wherein a temporal ratio of the stabilization phases (S) to the relief phases (R) relative to a reference duration is at least two to one or at least four to one and/or
wherein each stabilization phase (S) comprises at least two or at least four breaths.

13. A ventilator, wherein the ventilator comprises:
an actuation system for generating a respiratory air flow;
a respiratory air connection for providing the respiratory air flow;
a sensor system for recording at least one ventilation parameter with respect to a ventilation of a patient with the respiratory air flow provided by the respiratory air connection, the at least one ventilation parameter comprising a respiratory air pressure (p) applied to the respiratory air connection and/or a respiratory air volume (V) inhaled and/or exhaled by a patient;
the control unit of claim 1.

14. A computer program for operating the ventilator of claim 13, wherein the computer program comprises commands that prompt the control unit to carry out the following control method when the computer program is executed by the control unit:
receiving sensor data generated by the sensor system, which indicate an actual course of the at least one ventilation parameter (p, V);
generating a control signal for controlling the actuation system using the sensor data in order to adjust the respiratory air flow such that the actual course approaches a target course, wherein the target course comprises a plurality of stabilization phases (S), in which the actual course is intended to approach an upper limit (L1) in each case, and a plurality of relief phases (R), in which the actual course is intended to approach a lower limit (L2) in each case, wherein the stabilization phases (S) and the relief phases (R) alternate, wherein the upper limit (L1) is defined by a reference value for stabilizing lungs of the patient and to allow the patient to breathe spontaneously and is varied by repeatedly and alternately increasing and decreasing its value in relation to the reference value, in order to effect an additional carbon dioxide exhalation in each stabilization phase (S), the lower limit (L2) being defined in its value relative to the reference value in such a way that in each relief phase (R) an increased exhalation in comparison to the respective preceding stabilization phase (S) is effected to relieve the lungs of the patient.

15. A computer-readable medium on which the computer program of claim 14 is stored.
